(19) United States
(12) Patent Application Publication
(10) Pub. No.: US 2020/0372646 A1
(43) Pub. Date: Nov. 26, 2020

(54) MEASUREMENT APPARATUS FOR TWO TYPES OF BIOLOGICAL INFORMATION AND COMPUTER-READABLE STORAGE MEDIUM

(71) Applicant: CANON KABUSHIKI KAISHA, Tokyo (JP)

(72) Inventor: Hitoshi Furukawa, Suntou-gun (JP)

(21) Appl. No.: 16/966,349

(22) PCT Filed: Mar. 28, 2019
(86) PCT No.: PCT/JP2019/013512

§ 371 Date: Jul. 30, 2020

(30) Foreign Application Priority Data
Apr. 18, 2018 (JP) 2018-080162

(51) Int. Cl. G06T 7/00; G06T 5/20; A61B 5/024

(52) U.S. Cl. CPC G06T 7/0012; G06T 2207/30101; A61B 5/02416; G06T 5/20

(57) ABSTRACT

A measurement apparatus includes: a spectral sensor including, spectral means for dispersing reflected light from a living body or transmitted light that has passed through a living body according to a wavelength, and light receiving means including a plurality of pixels; detection means for detecting sample values indicating light reception results of the plurality of pixels at sampling timings; generation means for generating a biological signal relating to first biological information from sample values of a first pixel of the light receiving means at respective sampling timings; and determining means for determining a cycle of the biological signal. The detection means selects a sampling timing based on a cycle of the biological signal, and detects second biological information based on a sample value of at least one second pixel of the light receiving means at the selected sampling timing.

### TECHNICAL FIELD

The present invention relates to a measurement apparatus for measuring biological information and a computer-readable storage medium.

### BACKGROUND ART

A measurement apparatus is known that illuminates a portion of a living body with light and detects biological information by detecting an amount of light reflected from the living body or an amount of light that has passed through the portion of the living body. The biological information refers to various types of physiological/anatomical information of a living body such as a pulse rate, a degree of blood vessel stiffness, and a color and properties of skin, for example. For example, the pulse rate can be detected based on a pulse wave signal that indicates variation of an amount of reflected or transmitted light that is caused by blood movement inside a blood vessel.

Japanese Patent Laid-Open No. 2004-000467 discloses a pulse wave measuring apparatus, which is an example of the biological information measurement apparatus. According to Japanese Patent Laid-Open No. 2004-000467, the pulse wave measuring apparatus detects the pulse wave by illuminating a fingertip portion with a luminous flux and detecting a temporal change in the amount of reflected light. Japanese Patent Laid-Open No. 8-308634 discloses an apparatus for evaluating skin, which is an example of the measurement apparatus for biological information. According to Japanese Patent Laid-Open No. 8-308634, skin is evaluated by capturing an image of the skin surface and processing the captured image. Japanese Patent Laid-Open No. 2006-288842 discloses a configuration for obtaining a diameter of a blood vessel in an eyeground. According to Japanese Patent Laid-Open No. 2006-288842, because the diameter of a blood vessel changes according to a heartbeat, an image of the eyeground is captured in synchronization with a peak timing of a pulse wave signal that indicates the heartbeat.

Japanese Patent Laid-Open No. 2006-288842 discloses a configuration in which the timing at which the diameter of a blood vessel in an eyeground is measured is determined based on the pulse wave signal that indicates the change in the diameter of the blood vessel. That is, Japanese Patent Laid-Open No. 2006-288842 discloses a configuration in which the timing at which biological information, which is a diameter of a blood vessel, is measured is determined based on the change in the biological information over time. However, there are cases where the biological information to be measured is influenced by a change in another biological information over time. For example, when the spectral reflectance of skin is measured as a type of biological information for evaluating a state of the skin, the spectral reflectance of the skin may change due to blood pulsation, which is another type of biological information.

### SUMMARY OF INVENTION

The present invention is to provide a technology for suppressing, when biological information is measured, influence of another biological information that is different from the biological information to be measured.

According to an aspect of the present invention, a measurement apparatus includes: a spectral sensor including light emitting means for emitting light toward a measurement position, spectral means for dispersing reflected light from a living body at the measurement position or transmitted light that has passed through a living body at the measurement position according to a wavelength, and light receiving means including a plurality of pixels, each of which receives light including light having a predetermined wavelength that has been dispersed by the spectral means; detection means for detecting sample values indicating light reception results of the plurality of pixels at sampling timings; generation means for generating a biological signal relating to first biological information from sample values of a first pixel of the light receiving means at respective sampling timings; and determining means for determining a cycle of the biological signal, wherein the detection means selects a sampling timing based on a cycle of the biological signal, and detects second biological information based on a sample value of at least one second pixel of the light receiving means at the selected sampling timing.

Further features of the present invention will become apparent from the following description of exemplary embodiments with reference to the attached drawings.

### BRIEF DESCRIPTION OF DRAWINGS

FIG. 1 is a functional block diagram of a measurement apparatus according to an embodiment.

FIG. 2 is a diagram illustrating a hardware configuration of the measurement apparatus according to an embodiment.

FIG. 3A is a diagram illustrating a spectrum of a white light source according to an embodiment.

FIG. 3B is a diagram illustrating a configuration of a line sensor according to one embodiment.

FIG. 4 is a flowchart of processing for measuring biological information according to an embodiment.

FIG. 5A is a diagram illustrating a biological signal and a sampling timing to be selected according to an embodiment.

FIG. 5B is a diagram illustrating a biological signal and a sampling timing to be selected according to an embodiment.

FIG. 5C is a diagram illustrating a biological signal and a sampling timing to be selected according to an embodiment.

FIG. 6 is a flowchart of processing for measuring biological information according to an embodiment.

FIG. 7 is a diagram illustrating a biological signal and a sampling timing to be selected according to an embodiment.

### DESCRIPTION OF EMBODIMENTS

Hereinafter, illustrative embodiments of the present invention will be described with reference to the drawings. Note that the following embodiments are illustrative and do not limit the present invention to the contents of the embodiments. Also, in the following diagrams, constituent elements that are not required for describing the embodiments are omitted.

### First Embodiment

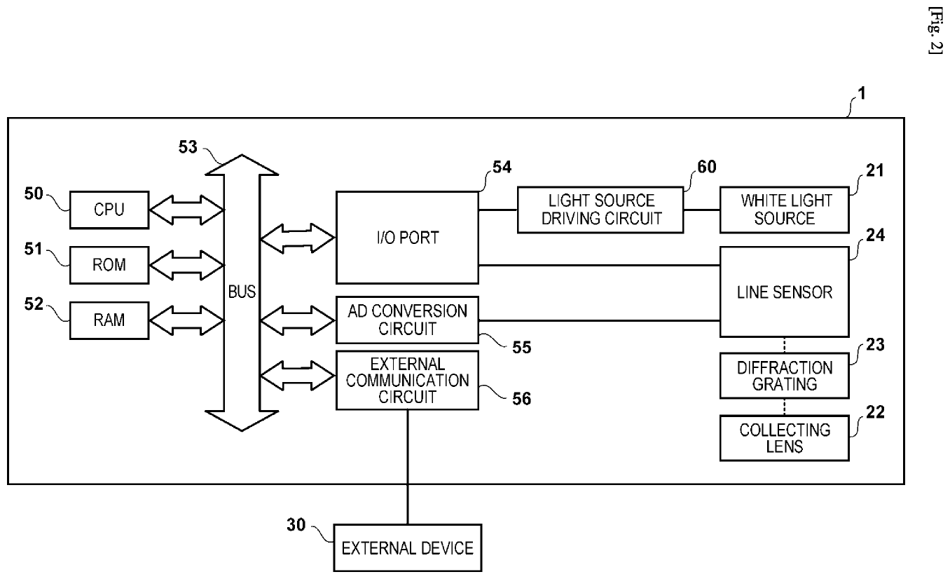

FIG. 2 is a diagram illustrating a hardware configuration of a measurement apparatus 1 according to the present embodiment. A CPU 50 is a control unit that performs overall control of the measurement apparatus 1. The CPU 50 executes later-described various types of control based on a program stored in a ROM 51. Note that the CPU 50 stores, to a RAM 52, data that is used when the various types of control are executed and data that needs to be temporarily stored. The CPU 50 can communicate with the ROM 51, the RAM 52, an I/O port 54, an AD conversion circuit 55, and an external communication circuit 56 via a bus 53. A light source driving circuit 60 controls light emission of the white light source 21. Also, the CPU 50 can control the light emission intensity of the white light source 21 by controlling the light source driving circuit 60 via the I/O port 54. As will be described later, the line sensor 24 receives reflected light of light emitted from the white light source 21 via a collecting lens 22 and a diffraction grating 23, and outputs a voltage corresponding to the received light amount to the AD conversion circuit 55. Then, the CPU 50 obtains the voltage corresponding to the received light amount that is output from the line sensor 24 via the AD conversion circuit 55. Moreover, the CPU 50 is configured to be able to communicate with an external device 30 via an external communication circuit 56. Note that the collecting lens 22, the diffraction grating 23, and the line sensor 24 constitute a spectral colorimeter (spectral sensor). Alternatively, the white light source 21, the collecting lens 22, the diffraction grating 23, and the line sensor 24 constitute a spectral sensor.

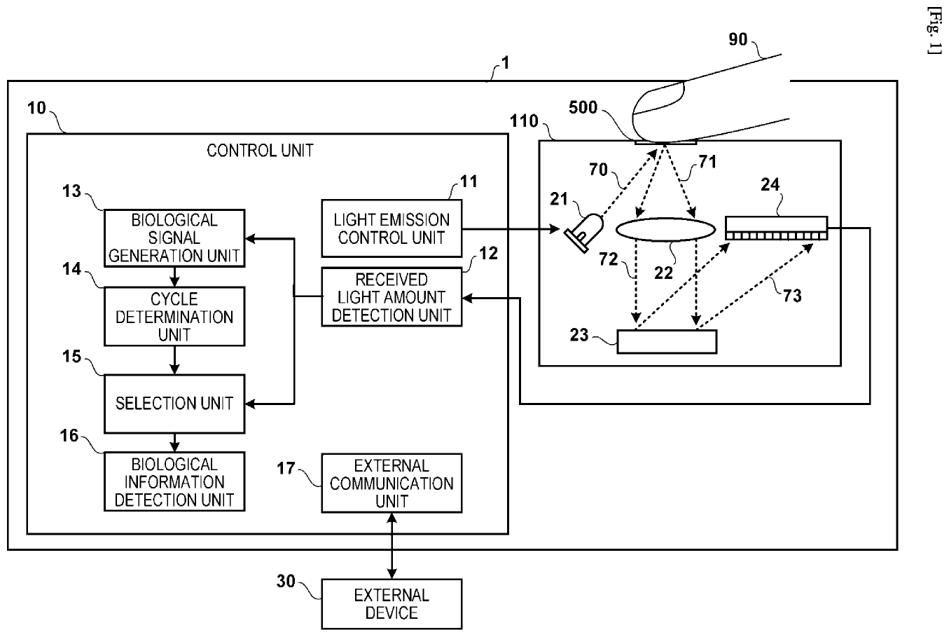

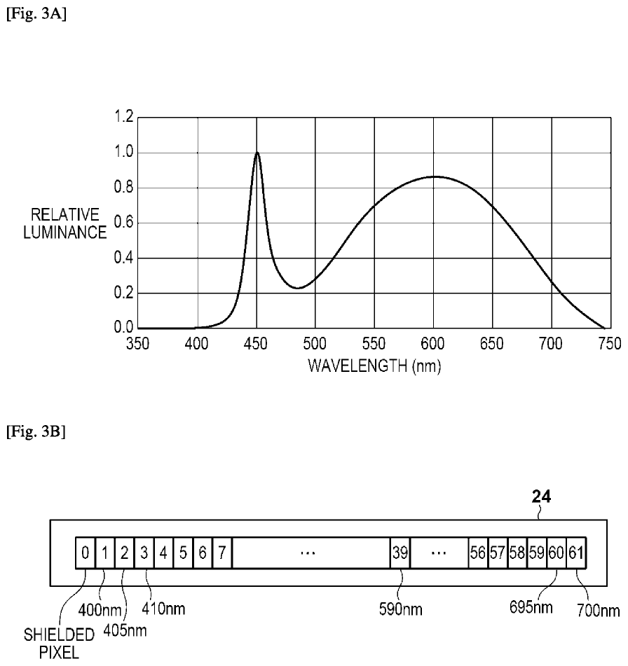

FIG. 1 is a block diagram illustrating operations of the measurement apparatus 1 in the present embodiment. The CPU 50 functions as a control unit 10 in FIG. 1, by executing a program stored in the ROM 51, in cooperation with the I/O port 54, the light source driving circuit 60, the AD conversion circuit 55, the external communication circuit 56, the ROM 51, and the RAM 52. A light emission control unit 11 corresponds to the CPU 50, the I/O port 54, and the light source driving circuit 60, adjusts the light emission intensity of the white light source 21, and controls the light emission of the white light source 21. The white light source 21 emits light having wavelength distribution that extends to the entirety of visible light. Tungsten light, a white LED, RGB (red, green, blue) three-color LEDs, or the like can be used as the white light source 21, for example. In the present embodiment, the white light source 21 is a white LED in which an LED element that emits blue light is packaged by a resin that includes a yellow fluorescent material. FIG. 3A shows relative intensity (luminance) of the white light source 21 used in the present embodiment for each wavelength. The peak at a wavelength of around 450 nm is a light emission spectrum of the blue LED, and the peak of around 600 nm is a spectrum of the yellow fluorescent material. This spectrum results from light that is emitted from a fluorescent material due to fluorescence upon receiving light from the LED element.

As shown in FIG. 1, light 70 emitted from the white light source 21 passes through a cover glass 500 provided in an aperture portion of the housing 110 at an angle of about 45 degrees relative to its normal direction and illuminates a fingertip 90, which is the measurement target, at a measurement position. Then, scattered light 71, which depends on the optical absorption property of the fingertip 90, is generated from the illumination light. A portion of the scattered light 71 is converted to parallel light 72 by the collecting lens 22, and the parallel light 72 is incident on the diffraction grating 23 at an incidence angle of 90 degrees. The diffraction grating 23 disperses the incident light according to the wavelength. The dispersed light 73 that has been dispersed is incident on pixels of the line sensor 24. Each pixel of the line sensor 24 outputs a voltage corresponding to the received light amount of the dispersed light 73 that has been incident thereon to a received light amount detection unit 12. FIG. 3B is a schematic diagram of the line sensor 24. The line sensor 24 according to the present embodiment includes 61 pixels needed to detect visible light in a wavelength range from about 400 nm to about 700 nm in units of 5 nm, and a shielded pixel on which light is not incident. In the present embodiment, the measurement apparatus 1 is calibrated and assembled such that a first pixel detects light including light having wavelength of about 400 nm, a 61^{th} pixel detects light including light having wavelength of about 700 nm, and 0^{th} pixel is the shielded pixel. The received light amount detection unit 12 corresponds to the CPU 50, the RAM 52, the AD conversion circuit 55, and the I/O port 54. In actuality, the AD conversion circuit 55 converts the voltage of each pixel that is output from the line sensor 24 to a 12-bit digital value, for example, and the CPU 50 obtains digital values indicating the received light amounts of the respective pixels from the AD conversion circuit 55. The line sensor 24 of the present embodiment is a charge accumulation type, and outputs a voltage signal, for each pixel, according to the light amount of the dispersed light that has been incident on the pixel during a predetermined accumulation time. This accumulation time is set by the CPU 50 to the line sensor 24 via the I/O port 54. Note that the line sensor 24 outputs a light reception result of each pixel at each sampling timing, and the AD conversion circuit 55 outputs a sample value that indicates the light reception result of each pixel at each sampling timing. Here, the intervals between adjacent sampling timings are an interval that is longer than the accumulation time of the line sensor 24. Also, the light reception results of the respective pixels at the respective sampling timings are stored in the RAM 52.

Note that, although not shown in FIG. 1, the measurement apparatus 1 is configured such that the measurement position is covered by a white reference plate when a fingertip 90, which is the measurement target, is not placed at the measurement position. Specifically, a white reference plate that can slide so as to cover the measurement position is provided, for example. Also, the configuration is such that, when a fingertip 90 is measured, the white reference plate is slid by the fingertip 90, and the fingertip 90 is placed at the measurement position. Note that a configuration may be adapted in which an elastic member such as a spring is connected to the white reference plate such that the white reference plate returns to the measurement position when the fingertip 90 is removed from the measurement position. In a state in which the measurement position is covered by the white reference plate 91, the line sensor 24 receives light according to the optical absorption property of the white reference plate.

The biological signal generation unit 13 generates a biological signal based on a light reception result of a predetermined pixel at each sampling timing, that is, sample values. In the present embodiment, a fingertip 90 of a living body is the measurement target, and the biological signal generation unit 13 generates the biological signal based on the sample values of a 39^{th} pixel that detects light including light having a wavelength of about 590 nm. This biological signal relates to the pulse, and is also referenced as a fingertip plethysmogram signal. The wavelength of about 590 nm that is used to generate the biological signal is a wavelength at which an amount of light that is absorbed by hemoglobin in a blood is relatively large. A cycle determination unit 14 determines the cycle of a biological signal that is generated by the biological signal generation unit 13. The pulse, which is first biological information, can be measured based on the cycle of the biological signal. That is, the biological signal is a signal for measuring the first biological information.

A selection unit 15 selects a sampling timing of a sample value to be used in the measurement of later-described second biological information based on the cycle of the biological signal determined by the cycle determination unit 14. The biological information detection unit 16 detects the second biological information based on the sample value at the sampling timing selected by the selection unit 15. Note that the sample value is stored in the received light amount detection unit 12, specifically in the RAM 52. In the present embodiment, the spectral reflectance of a fingertip 90 in a wavelength range from about 400 nm to about 700 nm is calculated as the second biological information in order to evaluate the state of skin. That is, the biological information detection unit 16 uses sample values of first to 61^{th} pixels of the line sensor for calculating spectral reflectances at the respective corresponding wavelengths. As described above, the second biological information is biological information that is different from the first biological information.

An external communication unit 17 corresponds to the external communication circuit 56, and communicates with the external device 30. The external device 30 instructs the measurement apparatus 1 to start and end measurement. Also, the measurement apparatus 1 transmits a biological signal corresponding to the first biological information and spectral reflectances at respective wavelengths corresponding to the second biological information to the external device 30. The external device 30 can calculate the pulse rate from the cycle of the biological signal. Moreover, the external device 30 can determine the degree of blood vessel stiffness based on the feature points of the biological signal and the values thereof. The external device 30 is a personal computer or a tablet terminal, for example. Note that the communication with the external device 30 may be wired communication or wireless communication.

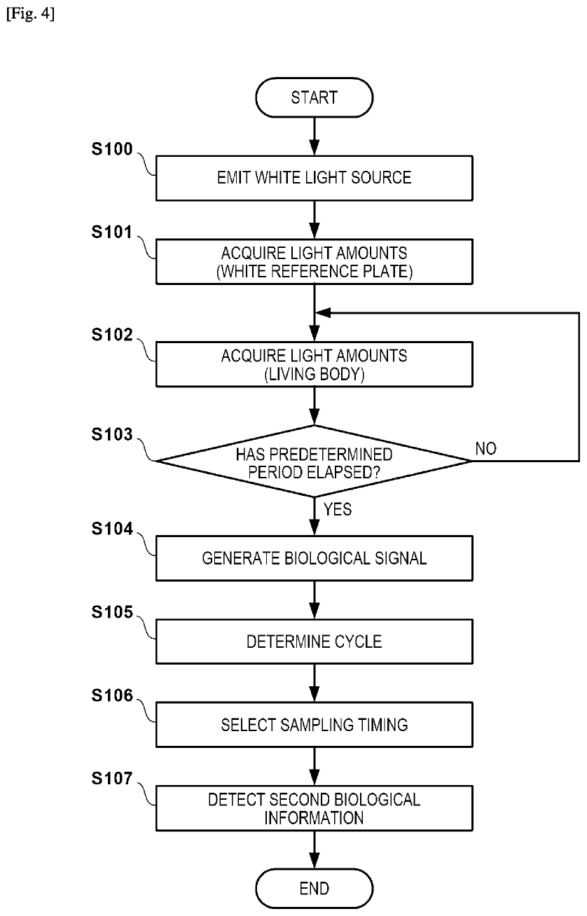

FIG. 4 is a flowchart of processing for detecting the biological information. Note that, when the processing in FIG. 4 is started, the white reference plate is assumed to be present at the measurement position. The light emission control unit 11, upon receiving an instruction to start measurement from the external device 30, causes the white light source 21 to emit light in step S100. In step S101, the received light amount detection unit 12 acquires received light amounts of all of the pixels in the line sensor 24 in a state in which the measurement position is covered by the white reference plate, and stores the received light amounts to the RAM 52. Note that these received light amounts correspond to the amount of light reflected by the white reference plate. Thereafter, the control unit 10 performs a display for prompting a user to place a fingertip 90, which is the measurement target, at the measurement position in a display unit of the external device. For example, when the user instructs to start processing from the external device 30 in a state in which the fingertip 90 is placed at the measurement position, the received light amount detection unit 12 acquires the received light amounts of all of the pixels in the line sensor 24, and stores the received light amounts to the RAM 52, in step S102. In step S103, the received light amount detection unit 12 determines whether or not a predetermined period has elapsed since the acquisition of the received light amounts in step S102 started, and continues the acquisition o f the received light amounts in step S102 until the predetermined period has elapsed. Here, the predetermined period is a period including a plurality of pulse cycles. For example, a normal pulse is about 60 beats per minute, and if a period including four pulse cycles is determined as the predetermined period, the predetermined period is four seconds. Note that, if the sampling interval of the received light amount for all of the pixels in the line sensor 24 is 50 ms, 80 sampling timings are included in four seconds, which is the predetermined period. Therefore, the control unit 10 obtains 80 sample values, for each pixel, indicating the received light amounts of the pixel during four seconds.

In step S104, the biological signal generation unit 13 generates a biological signal based on the sample values of the 0^{th} pixel and the 39^{th} pixel over the predetermined period that have been obtained in step S102. Note that if the sample value of the 0^{th} pixel and the sample value of the 39^{th} pixel that have been acquired at a sampling timing i are respectively denoted as R₀(i) and R₃₉(i), the amplitude B(i) of the biological signal at the sampling timing i is expressed as the following Equation (1).

B(i)=R₃₉(i)−R₀(i) (1)

Note that Equation (1) is for removing a dark component from the received light amount of the 39^{th} pixel that receives light including light having a wavelength of 590 nm based on the received light amount of the 0^{th} pixel. The biological signal generation unit 13 outputs the generated biological signal to the cycle determination unit 14.

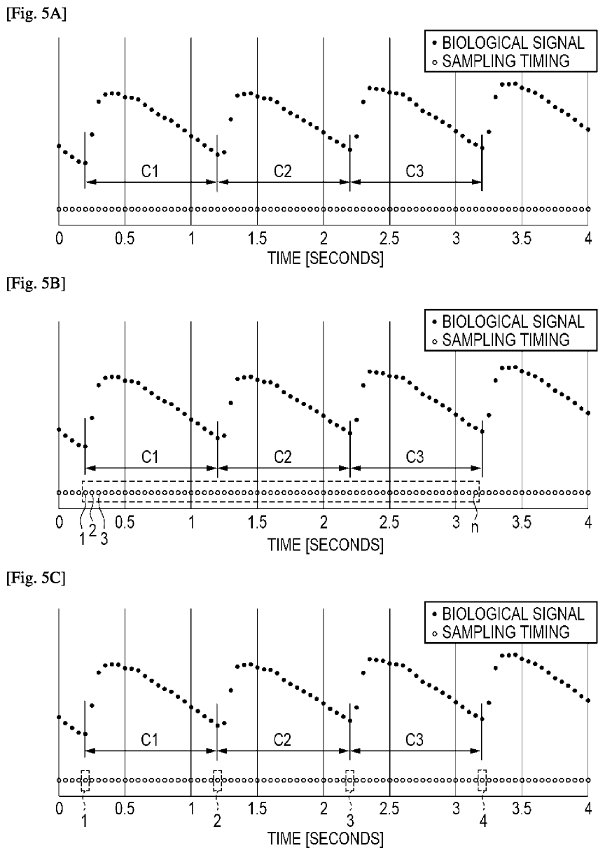

The cycle determination unit 14 determines the cycle of the biological signal in step S105. FIG. 5A shows the biological signal and determined cycles C1, C2, and C3. Note that white circles in FIG. 5A show sampling timings. In step S106, the selection unit 15 selects the sampling timing to be used in the calculation of the second biological information based on the cycle C of the biological signal. In the present embodiment, all of the sampling timings included in the three cycles C1, C2, and C3 of the biological signal are selected for each of the first to 61^{st} pixels. Note that the sampling timing at the boundary between the last cycle C3 and the next cycle shown in FIG. 5A is a fist sampling timing of the next cycle, and is not a sampling timing inside the cycle C3. For example, n times of sampling are assumed to have been performed in a period from the first sampling timing in the cycle C1 until the last sampling timing in the cycle C3, as shown in FIG. 5B. These timings are denoted as sampling timing i (i=1 to n) in time order. In this case, the selection unit 15 selects sampling timings from a sampling timing 1 to a sampling timing n.

In step S107, the biological information detection unit 16 detects the second biological information based on the sample values of the respective pixels at the sampling timings selected by the selection unit 15. Therefore, the biological information detection unit 16 averages, for each pixel, the sample values, that is the received light amounts, from the sampling timing 1 to the sampling timing n. For example, if the averaged received light amount of a k^{th} pixel is denoted as Aₖ, and the received light amount of the k^{th} pixel at a sampling timing i is denoted as Rₖ(i), Aₖ is obtained by the following equation.

Aₖ=(1/n)×Σ(Rₖ(i)−R₀(i)) (2)

Note that R₀(i) is a sample value, that is, a received light amount, of the 0^{th} pixel at the sampling timing i. Also, Σ in Equation (2) indicates a sum in a range of i=1 to n. Also, the reason for reducing R₀(i) in Equation (2) is to remove a dark component.

The biological information detection unit 16 obtains the spectral reflectance Sₖ of a pixel k (k=1 to 61) based on an averaged received light amount Aₖ of the pixel k using the following Equation (3).

Sₖ=(Aₖ/Wₖ)×Rₖ (3)

Note that Wₖ is a value obtained by reducing the received light amount of the 0^{th} pixel from the amount of light of the k^{th} pixel reflected from the white reference plate 91, which is obtained in step S101. Also, Rₖ denotes the spectral reflectance of the white reference plate 91 at the wavelength corresponding to the pixel number. For example, in this example, the k^{th} pixel receives light having a wavelength of (5 k+395) nm. Therefore, R₁ is the spectral reflectance of the white reference plate 91 at the wavelength of 400 nm. Note that Rₖ is stored in the ROM 51 in advance.

When the spectral reflectance of a living body is measured as the second biological information, the spectral reflectance of the living body changes due to the change in a blood flow in the living body. Therefore, the measurement result of the second biological information may vary depending on the sample value that is used to measure the second biological information. Also, when averaging processing or the like is performed on the sample values in order to reduce noise, or the like, the result of the averaging processing may vary depending on the relationship between the range of the sample values to be used in the averaging and the cycle of change in the blood flow in the living body. In the present embodiment, a pulse wave signal, that is, a biological signal indicating the change in the blood flow in the living body is generated, and the cycle of the biological signal is determined. Then, the sampling timings to be used to detect the second biological information is determined based on the determined cycle. For example, in the above-described example, the spectral reflectance is obtained by performing averaging processing, for each pixel, on sample values at sampling timings in a range corresponding to an integral multiplication of the cycle of the biological signal. Therefore, the influence of the change in the blood flow in the living body is cancelled, and the accuracy of measuring the second biological information can be improved. Also, in the present embodiment, the change in a biological signal, over time, indicating the first biological information, which is another biological information, is detected using the light emission control unit 11 and the received light amount detection unit 12 that are used to detect the second biological information, which is the measurement target. That is, a member for detecting the cycle of a biological signal need not be provided in addition to the members for detecting the second biological information, and therefore, the size of the measurement apparatus and the apparatus cost are prevented from increasing.

Also, the method of selecting the sampling timing by the selection unit 15 is not limited to the above-described method. For example, in the above-described embodiment, all the sampling timings in a range over three cycles of the biological signal are selected, but a configuration may be adopted in which sampling timings in a range over one cycle of the biological signal are selected, or sampling timings in a range over two or four or more cycles of the biological signal are selected. Also, the configuration may be such that sampling timings having a predetermined temporal positional relationship with respect to respective predetermined sampling timings in a plurality of cycles of the biological signal are selected. For example, in FIG. 5C, sampling timings corresponding to minimum values in amplitude in the respective cycles of the biological signal are selected. Note that, in FIG. 5C, four sampling timings are respectively selected from four cycles. Therefore, the spectral reflectance for each pixel is obtained by performing averaging processing, as described above, on the four sample values. Also, the configuration may be such that, a sampling timing a predetermined number after or before the sampling timing at which the amplitude is minimum is selected in each cycle, instead of the sampling timing at which the amplitude is minimum in each cycle, as shown in FIG. 5C. Also, the configuration may be such that the sampling timing, in each cycle, that is used to measure the second biological information is determined using a sampling timing at which the amplitude is maximum as a reference, instead of the sampling timing at which the amplitude is minimum in each cycle. Moreover, the configuration may be such that a sampling timing, in each cycle, at a temporal position at a predetermined phase is selected instead of a minimum value or a maximum value. Furthermore, the configuration may be such that the second biological information is determined based on one sampling timing in each cycle.

Note that the second biological information is not limited to the spectral reflectance of a living body. For example, the averaged received light amount Aₖ for each pixel may be the second biological information. Furthermore, when the skin color value is evaluated, chromaticity information in which the chromaticity (CIE L*a*b*) is obtained from the calculated spectral reflectance may be the second biological information. Specifically, tristimulus values XYZ are calculated from the calculated spectral reflectance using a color-matching function table in a range from 400 nm to 700 nm based on weighting factors in JIS Z 8722:2009. Then, L*a*b* can be calculated by performing chromaticity-conversion computation on the calculated XYZ. Also, all the pixels need not be used to measure the second biological information, and one or more pixels to be used in the measurement of the second biological information are determined according to the second biological information of the measurement target.

Note that, in the present embodiment, the amount of light reflected from the white reference plate 91 is detected in step S101 in FIG. 4, and Wₖ to be used in Equation (3) is obtained. However, a configuration may be adopted in which Wₖ is obtained and stored in the ROM 51 in advance, and in this case, the processing in step S101 can be omitted. Note that, in this case, the light emission intensity of the white light source 21 needs to be the same between when the white reference plate 91 is measured in advance and when the fingertip 90, which is the measurement target, is measured. This can be achieved by setting the value of current flowing through the white light source 21 to be the same between when the white reference plate 91 is measured in advance and when the fingertip 90 is measured.

### Second Embodiment

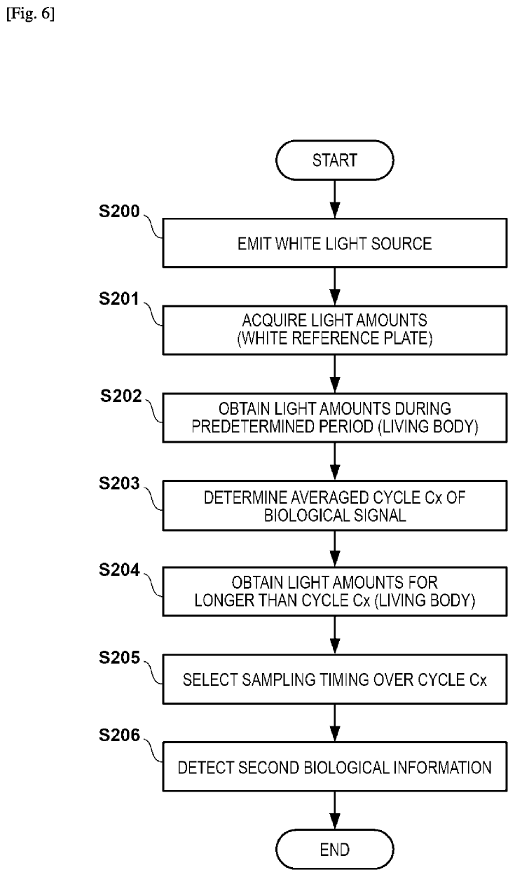

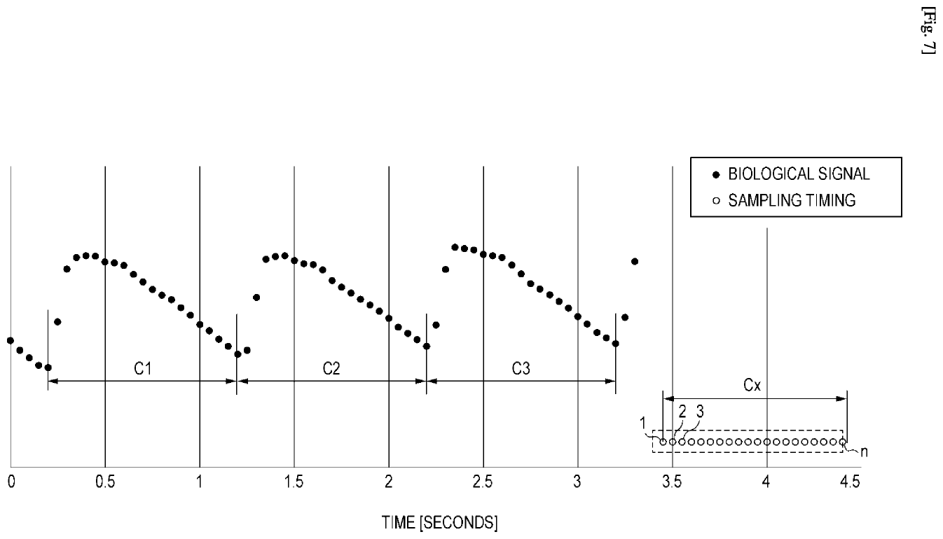

Next, a second embodiment will be described focusing on differences with the first embodiment. FIG. 6 is a flowchart of processing for detecting biological information in the present embodiment. The processing in steps S200 and S201 are similar to that in steps S100 and S101 in FIG. 4, and repetitive descriptions thereof are omitted. In step S202, a received light amount detection unit 12 obtains the amount of light reflected from a living body in a predetermined period. Note that the received light amount of 0^{th} and 39^{th} pixels need only be obtained in step S202. In step S203, similarly to steps S104 and S105 in FIG. 4, a biological signal generation unit 13 generates a biological signal, and a cycle determination unit 14 determines an average period of a predetermined number of consecutive cycles of the biological signal as an averaged cycle Cx. For example, as shown in FIG. 7, the cycle determination unit 14 determines three cycles C1, C2, and C3 of the biological signal, and the average period of the cycles C1, C2, and C3 is obtained as the averaged cycle Cx.

Thereafter, the received light amount detection unit 12 obtains the amount of light reflected from the living body in a period longer than the cycle Cx, in step S204. Note that, in step S204, the received light amounts of pixels to be used to detect second biological information are obtained. In step S205, a selection unit 15 selects sampling timings, of the sampling timings acquired in step S204, that extends over the cycle Cx, as shown in FIG. 7. Thereafter, similarly to the first embodiment, a biological information detection unit 16 detects the second biological information, in step S206.

As described above, in the present embodiment, the sample values of pixels needed to measure the second biological information need not be stored in the RAM 52 over a period for determining the cycle of the biological signal, and the needed capacity of the RAM 52 can be suppressed. Note that, in the present embodiment, the average value of a plurality of consecutive cycles of the biological signal is obtained, but the configuration may be such that the range of the sampling timings to be used to measure the biological information is determined based on the length of a specific cycle, instead of the average cycle. For example, when the biological information is measured, the heart rate of a person to be measured changes in a short period of time due to being strained or the like. Therefore, the configuration may be such that the cycle of the biological signal is determined in a predetermined period, or the cycle of the biological signal is determined in a period until the change in cycle of the biological signal decreases below a predetermined amount, and the range of the sampling timing to be used to detect the biological information is determined based on the length of the last cycle, for example.

In the present embodiment as well, similarly to the first embodiment, a plurality of sampling timings to be used to measure the second biological information are selected based on the cycle of the biological signal. According to this configuration, the measurement accuracy of the second biological information can be improved.

### Others

Note that, in the above embodiments, the line sensor 24 is configured to receive light reflected from a measurement target, but the line sensor 24 may be configured to receive transmitted light that has passed through a measurement target. Also, in the above embodiments, the pulse is measured as the first biological information, and the spectral reflectance and the chromaticity are measured as the second biological information, but the present invention is not limited to these pieces of biological information. For example, when a body motion at a fixed cycle due to respiration affects the measurement result of the second biological information, the configuration may be such that the sample values to be used to measure the second biological information are selected based on the change due to the cycle of the respiration.

### Other Embodiments

Embodiments of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiments and/or that includes one or more circuits (e.g., application specific integrated circuit (ASIC)) for performing the functions of one or more of the above-described embodiments, and by a method performed by the computer of the system or apparatus by, for example, reading out and executing the computer executable instructions from the storage medium to perform the functions of one or more of the above-described embodiments and/or controlling the one or more circuits to perform the functions of one or more of the above-described embodiments. The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-080162, filed on Apr. 18, 2018 which is hereby incorporated by reference herein in its entirety.

1. A measurement apparatus comprising:
a spectral sensor including a light emitting element configured to emit light toward a measurement position, a disperser configured to disperse reflected light from a living body at the measurement position or transmitted light that has passed through a living body at the measurement position according to a wavelength, and a light receiver including a plurality of pixels, each of which is configured to receive light including light having a predetermined wavelength that has been dispersed by the disperser;
a detector configured to detect sample values indicating light reception results of the plurality of pixels at sampling timings;
a generation unit configured to generate a biological signal relating to first biological information from sample values of a first pixel of the light receiver at respective sampling timings; and
a determining unit configured to determine a cycle of the biological signal, wherein
the detector is further configured to select a sampling timing based on a cycle of the biological signal, and detect second biological information based on a sample value of at least one second pixel of the light receiver at the selected sampling timing.

2. The measurement apparatus according to claim 1, wherein the detector is further configured to select all sampling timings included in one cycle of the biological signal, or all sampling timings included in a consecutive plurality of cycles of the biological signal.

3. The measurement apparatus according to claim 1, wherein the detector is further configured to select sampling timings having a predetermined temporal positional relationship with a predetermined sampling timing in each cycle of a consecutive plurality of cycles of the biological signal.

4. The measurement apparatus according to claim 3, wherein the predetermined sampling timing is a sampling timing corresponding to a maximum value or a minimum value of the biological signal in each cycle of the plurality of cycles.

5. The measurement apparatus according to claim 1, wherein the detector is further configured to select sampling timings at a predetermined phase in each cycle of a consecutive plurality of cycles of the biological signal.

6. The measurement apparatus according to claim 2, wherein the detector is further configured to detect the second biological information using an average value of sample values with respect to each of at least one second pixel at the selected sampling timings.

7. The measurement apparatus according to claim 1, further comprising a storage unit configured to store sample values of the plurality of pixels that are output from the spectral sensor,
wherein the detector is further configured to select a sampling timing of a sample value, which is stored in the storage unit, that is to be used to detect the second biological information from sampling timings inside the cycle that has been determined by the determining unit.

8. A measurement apparatus comprising:
a spectral sensor including a light emitting element configured to emit light toward a measurement position, a disperser configured to disperse reflected light from a living body at the measurement position or transmitted light that has passed through a living body at the measurement position according to a wavelength, and a light receiver including a plurality of pixels, each of which is configured to receive light including light having a predetermined wavelength that has been dispersed by the disperser;
a detector configured to detect sample values indicating light reception results of the plurality of pixels at sampling timings;
a generation unit configured to generate a biological signal relating to first biological information from sample values of a first pixel of the light receiver at respective sampling timings; and
a determining unit configured to determine an average value of a plurality of cycles of the biological signal, wherein
the detector is further configured to select a sampling timing based on the average value, and detect second biological information based on a sample value of at least one second pixel of the light receiver at the selected sampling timing.

9. The measurement apparatus according to claim 8, wherein detector is further configured to select sampling timings that extends over a period corresponding to the average value of the biological signal.

10. The measurement apparatus according to claim 9, wherein the detector is further configured to select sampling timings of sample values to be used to detect the second biological information from sampling timings that are temporally subsequent to the plurality of cycles that are used to determine the average value of the biological signal.

11. The measurement apparatus according to claim 8, wherein the detector is further configured to detect the second biological information using an average value of sample values at selected sampling timings with respect to each of the at least one second pixel.

12. The measurement apparatus according to claim 1, wherein the detector is further configured to obtain spectral reflectance at a wavelength, light having the wavelength being received by a second pixel, based on a sample value of the second pixel at the selected sampling timing.

13. The measurement apparatus according to claim 1, wherein the detector is further configured to obtain chromaticity of the living body based on a sample value of a second pixel at the selected sampling timing.

14. The measurement apparatus according to claim 1, wherein the biological signal is a pulse wave signal.

15. The measurement apparatus according to claim 1, wherein the detector is further configured to detect the second biological information after a change amount of the cycle of the biological signal that is determined by the determining unit has decreased below a predetermined amount.

16. A computer-readable storage medium storing a program, the program, upon being executed by one or more processors of a measurement apparatus including:
a spectral sensor including a light source configured to emit light toward a measurement position, a disperser configured to disperse reflected light from a living body at the measurement position or transmitted light that has passed through a living body at the measurement position according to a wavelength, and a light receiver including a plurality of pixels, each of which is configured to receive light including light having a predetermined wavelength that has been dispersed by the disperser;
a detector configured to detect sample values indicating light reception results of the plurality of pixels at sampling timings; and
the one or more processors,
causing the one or more processors to execute:
generating a biological signal relating to first biological information from sample values of a first pixel of the light receiver at respective sampling timings;
determining a cycle of the biological signal;
selecting a sampling timing based on a cycle of the biological signal; and
detecting second biological information based on a sample value of at least one second pixel of the light receiver at the selected sampling timing.

17. A computer-readable storage medium storing a program, the program, upon being executed by one or more processors of a measurement apparatus including:
a spectral sensor including a light source configured to emit light toward a measurement position, a disperser configured to disperse reflected light from a living body at the measurement position or transmitted light that has passed through a living body at the measurement position according to a wavelength, and a light receiver including a plurality of pixels, each of which is configured to receive light including light having a predetermined wavelength that has been dispersed by the disperser;
a detector configured to detect sample values indicating light reception results of the plurality of pixels at sampling timings; and
the one or more processors,
causing the one or more processors to execute:
generating a biological signal relating to first biological information from sample values of a first pixel of the light receiver at respective sampling timings;
determining an average value of a plurality of cycles of the biological signal;
selecting a sampling timing based on the average value; and
detecting second biological information based on a sample value of at least one second pixel of the light receiver at the selected sampling timing.
